(19) United States
(12) Patent Application Publication
(10) Pub. No.: US 2023/0075170 A1
(43) Pub. Date: Mar. 9, 2023

(54) NOVEL SALTS OF NILOTINIB AND POLYMORPHIC FORMS THEREOF

(71) Applicant: CIPLA LIMITED, Mumbi, Maharashtra (IN)

(72) Inventors: Srinivas Laxminarayan PATHI, Bangalore (IN); Ramanaiah CHENNURU, Nellore(Dt) Andhra Pradesh (IN); Ravikumar PUPPALA, Bangalore (IN); Raju BARLA, Telangana (IN); Krishna RAGHAVENDRA, Bangalore East (IN)

(21) Appl. No.: 17/798,742

(22) PCT Filed: Feb. 15, 2021
(86) PCT No.: PCT/IN2021/050142

§ 371 Date: Aug. 10, 2022

(30) Foreign Application Priority Data
Feb. 15, 2020 (IN) 202021006617

(51) Int. Cl. C07D 401/14

(52) U.S. Cl. CPC C07D 401/14; C07B 2200/13

(57) ABSTRACT

The present invention relates to the solid forms of Nilotinib D-Malate and Nilotinib D-Tartrate, processes for preparation thereof, pharmaceutical compositions thereof, and methods of use thereof.

### TECHNICAL FIELD

The present invention relates to the solid forms of Nilotinib D-Malate and Nilotinib D-Tartrate, processes for preparation thereof, pharmaceutical compositions thereof, and methods of use thereof.

### BACKGROUND

Tasigna (nilotinib) is a kinase inhibitor indicated for the treatment of adult and pediatric patients greater than or equal to 1 year of age with newly diagnosed of Philadelphia chromosome-positive chronic myeloid leukemia (Ph+CML) in chronic phase. Tasigna is approved in the form of 50, 150 and 200 mg capsules. Tasigna capsules contain nilotinib as a monohydrate monohydrochloride. Tasigna capsules also contain various inactive ingredients, including magnesium stearate and polyoxamer 188. The prescribing information recommends that Tasigna should be taken on an empty stomach and that no food be consumed for 2 hours before and 1 hour after the dose is taken. The prescribing information also recommends that, for patients unable to swallow capsules, the capsule contents may be dispersed in applesauce.

Nilotinib, is chemically termed as 4-methyl-N-[3-(4-methyl-1H-imidazol-1-yl)-5-(trifluoromethyl) phenyl]-3-[[4-(3-pyridinyl)-2-pyrimidinyl]amino]-benzamide, having the following formula I:

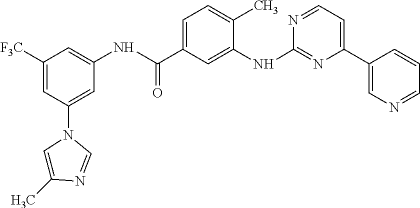 [SMILES: Cc1cn(-c2cc(NC(=O)c3ccc(C)c(Nc4nccc(-c5cccnc5)n4)c3)cc(C(F)(F)F)c2)cn1]

Nilotinib and its process of preparation are disclosed in U.S. Pat. No. 7,169,791 B2.

Prior art suggests that in addition to the free base and hydrochloride salt, Nilotinib exist in the form of a number of other salts. For example, in U.S. Pat. Nos. 8,163,904 B2, 8,937,082 B2, 9,090,598 B2, 10,138,221 B2, WO 2007/105870 A1, WO 2015/092624 A1, WO 2018/076117 A1, IP.COM 2010, 10(7B), 3,IP. COM 2010, 10 (9A), 21, and IP. COM 2010, 10 (12A), 18.

Nilotinib and its salts can exist in different polymorphic forms, which may differ from each other in terms of stability, physical properties, spectral data and methods of preparation and thus the bioavailability of the same also varies with polymorphic modification.

Thus, there is a need in the art to provide a pharmaceutically active substance which not only is characterized by high pharmacological potency but also satisfies the above-mentioned physicochemical requirements as far as possible.

New or improved salt/forms of existing kinase inhibitors are continually needed for developing new, improved and more effective pharmaceutical formulations for the treatment of cancer and other diseases. The salt/forms and methods of preparing the salt forms described herein are directed toward these needs and other ends.

### SUMMARY OF THE INVENTION

The present invention provides, inter alia, salts of Nilotinib of Formula I.

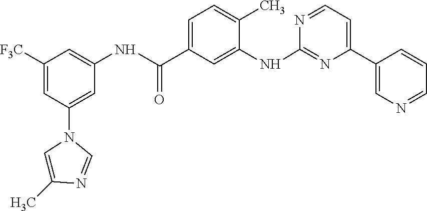 [SMILES: Cc1cn(-c2cc(NC(=O)c3ccc(C)c(Nc4nccc(-c5cccnc5)n4)c3)cc(C(F)(F)F)c2)cn1]

In one aspect, the present invention provides pharmaceutically acceptable acid addition salts selected from:

Nilotinib D-malic acid salt; and Nilotinib D-tartaric acid salt, or a solvate or hydrate; co-crystal thereof.

The present invention further provides methods of preparing a salt of the invention comprising steps of combining Nilotinib free base with an acid selected from D-malic acid, or D-tartaric acid, in a suitable solvent, and thereafter optionally isolating the acid addition salt so formed.

The present invention further provides one or more polymorphic forms or pseudo polymorphic forms or co-crystals of the salts of Nilotinib. Accordingly, pseudo polymorphs provided include hydrates and/or solvates.

The present invention further provides premixes of acid salts of Nilotinib and polymorphic forms thereof.

The present invention further provides methods of preparing premixes of acid salts of Nilotinib and polymorphic forms thereof.

The present invention further provides substantially pure an acid salt of Nilotinib (which may be present in crystalline or amorphous form), or solvate or hydrate or co-crystals or premixes thereof, having a purity of greater than 99.5% wherein process related impurities are below 0.3% by HPLC.

The present invention further provides pharmaceutical compositions comprising an acid salt of Nilotinib (which may be present in crystalline or amorphous form), or solvate or hydrate or co-crystals or premixes thereof; and at least one pharmaceutically acceptable excipient.

The present invention further provides methods of modulating an activity of tyrosine kinase comprising contacting tyrosine kinase with a salt or solvate or hydrate or co-crystals or premixes thereof of the invention.

The present invention further provides methods of treating a disease in a patient, wherein the disease is associated with Philadelphia chromosome-positive chronic myeloid leukemia (Ph+CML), comprising administering to the patient a therapeutically effective amount of a salt or solvate or hydrate or co-crystals or premixes thereof of the invention.

The present invention further provides methods of treating Philadelphia chromosome-positive chronic myeloid leukemia (Ph+CML) in a patient, comprising administering to the patient a therapeutically effective amount of a salt or solvate or hydrate or co-crystals or premixes thereof of the invention.

### BRIEF DESCRIPTION OF THE DRAWINGS

FIG. 1 shows an X-ray powder diffraction (XRPD) pattern of Nilotinib D-malate Form C1

FIG. 2 shows a Differential Scanning calorimetry thermogram (DSC) of Nilotinib D-malate Form C1

FIG. 3 shows a Thermogravimetric analysis (TGA) of Nilotinib D-malate Form-C1

FIG. 4 shows an X-ray powder diffraction (XRPD) pattern of Nilotinib D-tartrate Form C1 from example-2

FIG. 5 shows an X-ray powder diffraction (XRPD) pattern of Nilotinib D-tartrate Form C1 from examples-3, 5 and 6

FIG. 6 shows a Differential Scanning calorimetry thermogram (DSC) of Nilotinib D-tartrate Form C1

FIG. 7 shows a Thermogravimetric analysis (TGA) of Nilotinib D-tartrate Form C1

### DETAILED DESCRIPTION

The present invention provides, inter alia, novel acid salts of the tyrosine kinase inhibitor Nilotinib of formula I

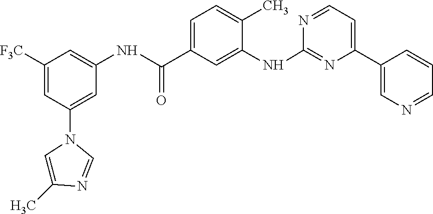 [SMILES: Cc1cn(-c2cc(NC(=O)c3ccc(C)c(Nc4nccc(-c5cccnc5)n4)c3)cc(C(F)(F)F)c2)cn1]

selected from the D-malic acid salt, and D-tartaric acid salt or a solvate or hydrate or co-crystals or premixes thereof. These salts modulate the activity of one or more tyrosine kinase and are useful, for example, in the treatment of diseases associated with tyrosine kinase expression or activity.

Salts of the present invention also include all isotopes of atoms occurring in the salts. Isotopes include those atoms having the same atomic number but different mass numbers. For example, isotopes of hydrogen include tritium and deuterium.

The salts of the invention have numerous advantageous properties over the free base form and other salt forms. In particular, these salts were highly crystalline which would facilitate the preparation of pharmaceutical formulations and improve general handling, manipulation, and storage of the active ingredient. The salts of the invention also have superior aqueous solubility, rate of dissolution, chemical stability (with a longer shelf life), compatibility with excipients, and reproducibility compared with the free base form.

In some embodiments, the salts of the invention are substantially isolated. By “substantially isolated” is meant that the salt is at least partially or substantially separated from the environment in which it was formed or detected. Partial separation can include, for example, a composition enriched in the salt of the invention. Substantial separation can include compositions containing at least about 50%, at least about 60%, at least about 70%, at least about 80%, at least about 90%, at least about 95%, at least about 97%, or at least about 99% by weight of the salt

Salts of the invention can be prepared using known techniques. Conventionally, a salt form is prepared by combining in solution the free base compound and an acid containing the anion of the salt form desired, and then isolating the solid salt product from the reaction solution by processes known to the chemist (e.g., by crystallization, precipitation, evaporation, etc.). Other salt-forming techniques can be employed.

The use of certain solvents during the process has been found to produce different polymorphic forms of the acid salts of Nilotinib, which may exhibit one or more favourable characteristics described above. The processes for the preparation of the polymorphs described herein, and characterization of these polymorphs are described in greater detail below.

As used herein, the term “solvated” is understood to mean formation of a complex of variable stoichiometry comprising Nilotinib salt and a solvent. Such solvents for the purpose of the invention may not interfere with the biological activity of the solute. Typically, the solvent used is a pharmaceutically acceptable solvent. Examples of suitable pharmaceutically acceptable solvents include C1-C4 alcohol solvents, methylene dichloride (MDC) and tetrahydrofuran (THF) and solvates other than water at levels of more than 1%. The solvate can be isolated either as an amorphous form or in a crystalline form, preferably in crystalline form.

The solvate can be further isolated either in anhydrous form or hydrated form.

As used herein, the term “hydrate” is understood as a substance that is formed by adding water molecules. The skilled person will appreciate that the water molecules are absorbed, adsorbed or contained within a crystal lattice of the solid compounds, usually in defined stoichiometric ratio. The notation for a hydrated compound may be. nH₂O, where n is the number of water molecules per formula unit of the compound. For example, in a hemihydrate, n is 0.5; in a monohydrate n is one; in a sesquihydrate, n is 1.5; in a dihydrate, n is 2; and so on.

As used herein, the term “substantially the same X-ray powder diffraction pattern” is understood to mean that those X-ray powder diffraction patterns having diffraction peaks with 2θ values within ±0.2° of the diffraction pattern referred to herein are within the scope of the referred to diffraction pattern.

According to one aspect of the present invention, there is provided novel acid salt of Nilotinib, Nilotinib D-malate. The Nilotinib D-malate may be in the form of a derivative thereof. The derivative may be a pharmaceutically acceptable solvate, hydrate, tautomer, anhydrate, complex, polymorph, prodrug or combination thereof.

The D-malate salt may be isolated in pseudo polymorphic form as a solvate optionally in hydrated form, or as a non-hydrated solvate.

Because D-malic acid has two acid groups per molecule, compounds of these acids and Nilotinib may be isolated as either mono- or di-acid addition salts and/or solvates thereof having either one or two Nilotinib molecules per acid molecule respectively.

The ratio of Nilotinib to D-malic acid may range from about 1 to about 2 molecules of Nilotinib per 1 molecule of D-malic acid. Preferably, the ratio is 1 molecule of Nilotinib per 1 molecule of D-malic acid. Thus, D-malate salt is mono D-malate salt of Nilotinib of formula I.

In a further aspect, the present invention relates to a process for preparing Nilotinib D-malate which comprises reacting the free base of Nilotinib with D-malic acid, wherein molar ratio of Nilotinib to D-malic acid is 1:0.95 to 1:1.1.

The D-malate salt according to the invention is characterised by good crystallinity and low amorphisation during grinding and compression. In addition, it is not hygroscopic and is readily soluble in physiologically acceptable solvents.

The inventors have discovered a surprisingly advantageous crystalline polymorphic form of Nilotinib D-malate which hereinafter referred to as “Form C1”. This form is described in more detail herein.

The crystalline Nilotinib D-malate Form C1 is relatively stable towards moisture and humidity, thereby representing a crystalline form of Nilotinib, thus enhancing the efficacy of the parent molecule in lower doses.

The crystalline Nilotinib D-malate Form C1 according to the present invention may be characterized by powder X-ray diffraction.

Crystalline Nilotinib D-malate Form C1 may be characterized by having an XRPD diffractogram comprising peaks at 4.92, 9.86, 15.23, 16.95, 18.80 and 21.32±0.2 °2θ. The XRPD diffractogram may comprise further peaks at 6.82, 13.35 and 13.83±0.2 °2θ.

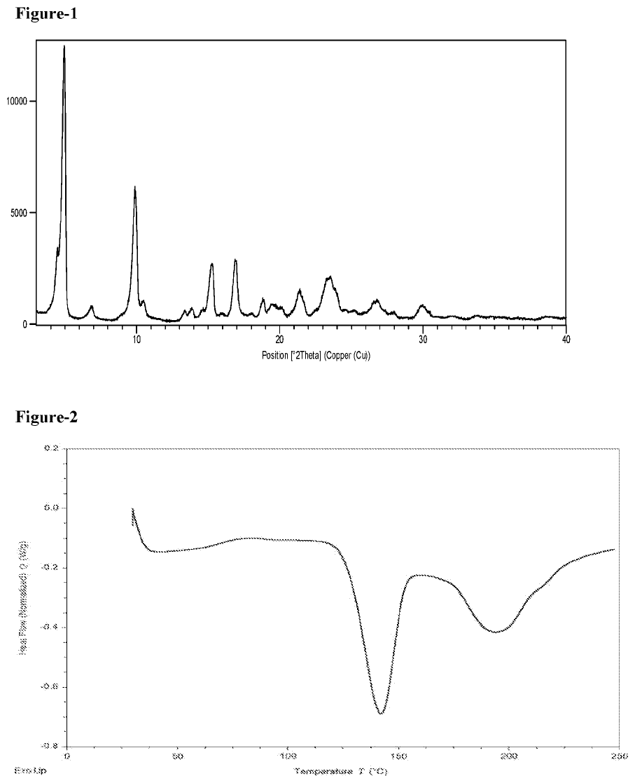

The XRPD diffractogram may be as depicted in FIG. 1.

Crystalline Nilotinib D-malate Form C1 may be characterized by having a DSC thermogram as shown in FIG. 2.

The DSC plot for the sample shows an endotherm peak melting with an onset at 127.39° C., a peak maximum at 141.97±5° C.

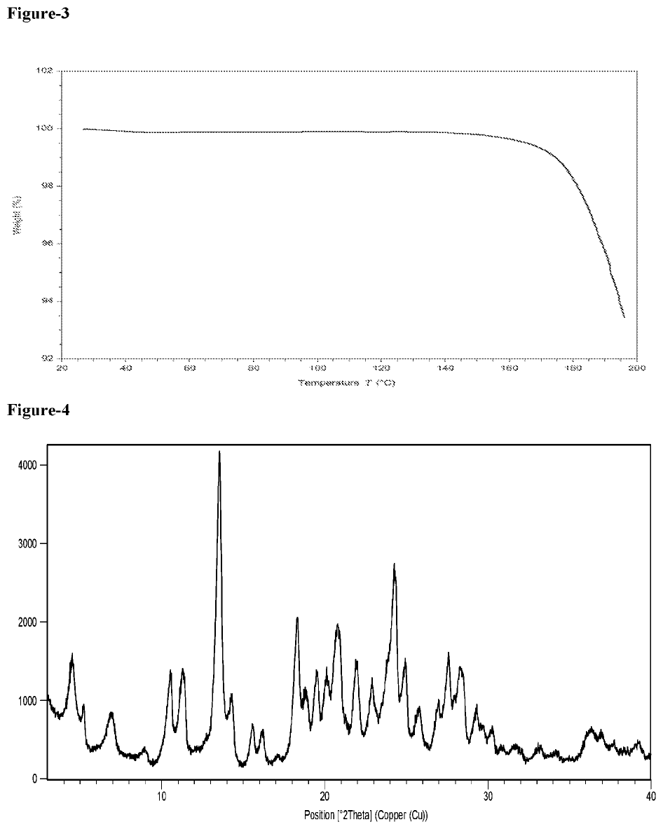

Crystalline Nilotinib D-malate Form C1 may be characterized by having a TGA thermogram substantially as depicted in FIG. 3.

TGA data indicated a weight loss of approximately 0.289% at temperature about 160° C.

According to second aspect of the present invention, there is provided Nilotinib D-tartrate.

The Nilotinib D-tartrate may be in the form of a derivative thereof. The derivative may be a pharmaceutically acceptable solvate, hydrate, ester, tautomer, anhydrate, complex, polymorph, prodrug, co-crystal or combination thereof.

The D-tartrate salt may be isolated in pseudo polymorphic form as a solvate optionally in hydrated form, or as a non-hydrated solvate.

Because D-tartaric acid has two acid groups per molecule, compounds of these acids and Nilotinib may be isolated as either mono- or di-acid addition salts and/or solvates thereof having either one or two Nilotinib molecules per acid molecule respectively.

The ratio of Nilotinib to D-tartaric acid may range from about 1 to about 2 molecules of Nilotinib per 1 molecule of D-tartaric acid. Preferably, the ratio is 1 molecule of Nilotinib per 1 molecule of D-tartaric acid. Thus, D-tartrate salt is mono D-tartrate salt of Nilotinib of formula I

In a further aspect, the present invention relates to a process for preparing Nilotinib D-tartrate which comprises reacting the free base of Nilotinib with D-tartaric acid wherein molar ratio of Nilotinib to D-malic acid is 1:0.95 to 1:1.1.

The D-tartrate salt according to the invention is characterised by good crystallinity and low amorphisation during grinding and compression. In addition, it is not hygroscopic and is readily soluble in physiologically acceptable solvents.

The inventors have discovered a surprisingly advantageous crystalline polymorphic form of Nilotinib D-tartrate which hereinafter referred to as “Form C1”. This form is described in more detail herein.

The crystalline Nilotinib D-tartrate Form C1 is relatively stable towards moisture and humidity, thereby representing a crystalline form of Nilotinib, thus enhancing the efficacy of the parent molecule in lower doses.

The crystalline Nilotinib D-tartrate Form C1, according to the present invention may be characterized by powder X-ray diffraction.

Crystalline Nilotinib D-tartrate Form C1 may be characterized by having an XRPD comprising peaks at 6.88, 10.47, 11.24, 13.50, 18.28 and 24.17±0.2 °2θ. The XRPD diffractogram may comprise further peaks at 15.57, 19.48, 20.77 and 21.92±0.2 °2θ.

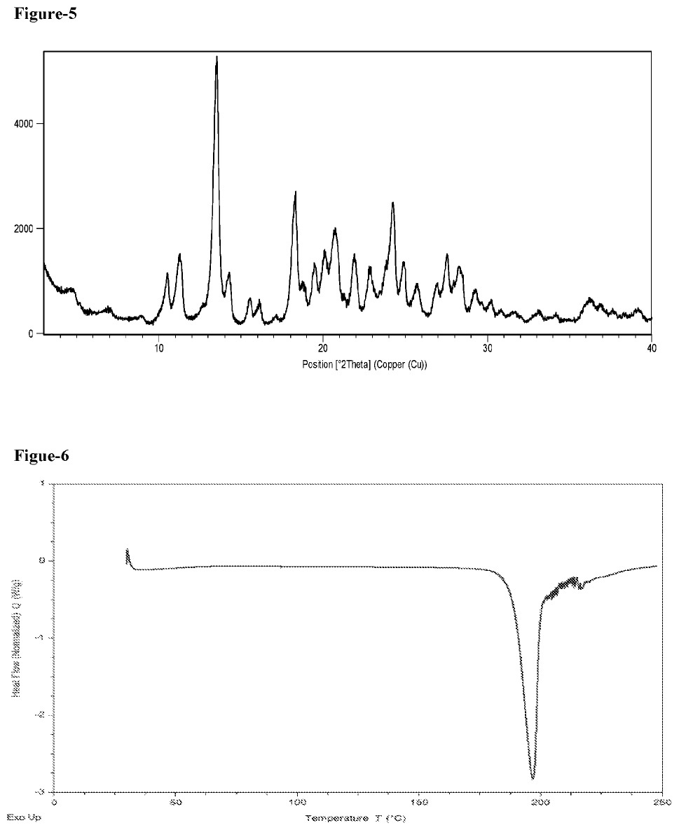

The XRPD diffractogram may be as depicted in FIG. 4 and FIG. 5.

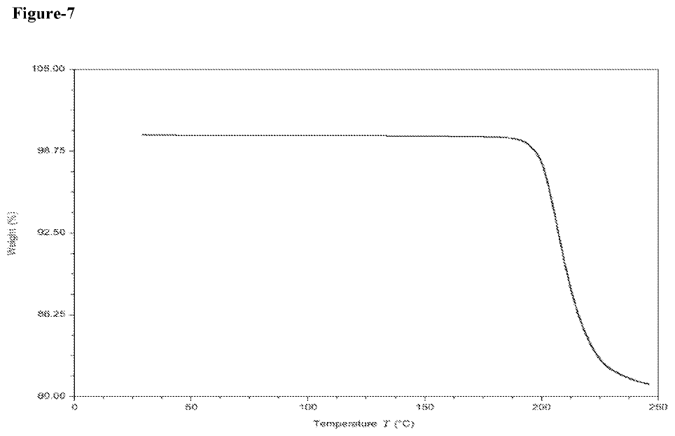

Crystalline Nilotinib D-tartrate Form C1 may be characterized by having a DSC thermogram as shown in FIG. 6.

The DSC plot for the sample shows an endotherm peak melting with an onset at 189.31° C., a peak maximum at 196.98±5° C.

Crystalline Nilotinib D-tartrate Form C1 may be characterized by having a TGA thermogram substantially as depicted in FIG. 7.

TGA data indicated a weight loss of approximately 0.284% at temperatures about 190° C.

Those skilled in the art would recognize that these crystal form or amorphism can be identified by a variety of technical means, including, but not limited to infrared absorption spectroscopy (IR), melting point method, Nuclear magnetic resonance (NMR), Raman spectroscopy, dynamic vapor sorption (DVS), X-ray single crystal diffraction, dissolution calorimetry, scanning electron microscopy (SEM), quantitative analysis, solubility, dissolution rate and a combination thereof. Further bulk and tapped density of the polymorphic forms may also be evaluated.

Nilotinib base as used in any of the processes described in the present application may be prepared according to U.S. Pat. No. 7,169,791, Example 92, which is incorporated herein by reference.

The Nilotinib, as free base, may be in any polymorphic form or in a mixture of any polymorphic forms.

The Nilotinib may be converted to an acid addition salt either by first isolating the free base or without isolating the free base. In one aspect, Nilotinib is not isolated, i.e. the free base is converted to an acid salt in situ.

In one aspect, Nilotinib is dissolved in a suitable solvent to facilitate formation of the acid salt. Examples of suitable solvents include, but are not limited to, alcohols such as methanol, ethanol, isopropanol, n-propanol, isobutanol, n-butanol, 1,2-dimethoxy ethanol, 2-methoxy ethanol, 2-ethoxy ethanol and ethylene glycol, and like or mixtures thereof; ethers such as tetrahydrofuran, diethyl ether, isopropyl ether, diisopropyl ether (DIPE), 1,4-dioxane, methyl tertiary butyl ether (MTBE), and like; ketones such as acetone, methyl ethyl ketone (MEK), methyl isobutyl ketone (MIBK); aprotic polar solvents such as dimethyl formamide (DMF), dimethyl acetamide (DMA), dimethyl sulfoxide (DMSO), N-methyl-2-pyridone, N-methyl-2-pyrrolidone, 1-methyl pyrolidinone (NMP) or mixtures thereof; esters like methyl acetate, ethyl acetate and isopropyl acetate; chlorinated solvents like chloroform, dichloromethane, nitriles like acetonitrile, hydrocarbons such as heptane, hexane, benzene, toluene, xylene and the like or mixtures thereof.

The solution containing Nilotinib is treated with D-malic acid, or D-tartaric acid. The acid may be in the form of a solution or solid. The resulting acid addition salt may be isolated as a solid by any known technique, including but not limited to, cooling, chilling, completely or partially distilling solvents, and/or filtering.

The reaction may be carried out at elevated temperature to facilitate the reaction. The salts may be isolated from the reaction mixture by any of the general techniques known in the art

Alternatively, acid addition salts of Nilotinib may be prepared in accordance with the present invention by a salt interconversion method. This process involves reacting an acid salt of Nilotinib with a suitable base to form the free base of Nilotinib and thereafter converting the free base so formed into an acid salt form (by addition of a different acid).

The obtained acid addition salts of Nilotinib such as Nilotinib D-malate and Nilotinib D-tartrate may be further purified.

According to another aspect of the present invention, there is provided substantially pure Nilotinib D-malate and Nilotinib D-tartrate having a purity of at least 95%, preferably at least 99%, more preferably at least 99.5% area % by HPLC.

Preferably, the purified salts Nilotinib of formula I contain about 1% or less area by HPLC %, of the genotoxic impurity 3-(trifluoromethyl)-5-(5-methyl-1H-imidazol-1-yl)benzenamine (‘5 methyl isomer’) of the following formula

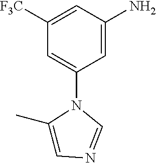 [SMILES: Cc1cncn1-c1cc(N)cc(C(F)(F)F)c1]

The obtained Nilotinib salts more preferably contain about 0.10% or less by area on HPLC, of the 5-methyl isomer impurity, most preferably, about 0.01% or less by area on HPLC, of the 5-methyl isomer impurity.

Crystalline forms of the present invention may be prepared by dissolving, crystallizing, stirring, evaporating the solvent or seeding with crystal. The crystals may be isolated form the reaction mixture by any of the general techniques known in the art.

In certain aspects, the acid salts and polymorphic forms described herein may potentially exhibit improved properties. For example, in certain aspects, the acid salts and polymorphic forms described herein may potentially exhibit improved long term physical and chemical stability. Such improved stability could have a potentially beneficial impact on the manufacture of the Nilotinib, such as for example offering the ability to store process intermediate for extended periods of time. Improved stability could also potentially benefit a composition or pharmaceutical composition of the Nilotinib. In further aspects, the salts and polymorphic forms described herein may also potentially result in improved yield of the Nilotinib, or potentially result in an improvement of the quality of the Nilotinib. In certain aspects, the salts and polymorphic forms described herein may also exhibit improved pharmacokinetic properties and/or potentially improved bioavailability.

The acid salts and polymorphic forms of the present invention may be administered by any route appropriate to the condition to be treated. Suitable administration routes include, but are not limited to, oral, rectal, nasal, pulmonary, topical, vaginal and parenteral.

The pharmaceutical compositions of the present invention comprise a Nilotinib or a pharmaceutically acceptable acid addition salt thereof of the type disclosed herein, together with one or more pharmaceutically acceptable excipients, and optionally one or more further active pharmaceutical ingredients.

In one aspect, the pharmaceutical composition of the present invention is formulated to provide immediate release of the active pharmaceutical ingredient(s) present therein. In an alternative aspect, the pharmaceutical composition of the present invention is formulated to provide controlled release of the active pharmaceutical ingredient(s) present therein. Controlled release comprises delayed, sustained and pulsed release of the active pharmaceutical ingredient(s).

Suitable pharmaceutical excipients are known in the art and include, but are not limited to, carriers, diluents and/or vehicles. The excipients (s) must be “acceptable” in the sense of being compatible with the other ingredients of the pharmaceutical composition and not harmful to the patient. The excipient(s) may be selected to provide a desired release profile of the active pharmaceutical ingredient(s) present.

The pharmaceutical compositions of the present invention comprise a therapeutically effective amount of Nilotinib or a pharmaceutically acceptable acid addition salt thereof. Suitable dosages include, but are not limited to, 150 mg, and 200 mg.

In a further aspect, the present invention provides, premixes of acid salts of Nilotinib and polymorphic forms thereof and process for the preparation of the same.

Within the context of the present invention, premixes of acid salts of Nilotinib and polymorphic forms thereof of the present invention may be prepared by the steps of:

a) dissolving acid salts of Nilotinib or polymorphic forms thereof in a solvent to form a solution;

b) combining the solution with one or more pharmaceutically acceptable excipients; and

c) removing the solvent to isolate a premix of acid salts of Nilotinib and polymorphic forms thereof.

Within the context of the methods of generating premixes of acid salts of Nilotinib and polymorphic forms thereof, the pharmaceutically acceptable excipient may be dissolved in a second solvent to form a second solution. The first and the second solvents used in these methods may be the same or different.

The solvents used in the methods of the present invention may be selected from the group consisting of alcohol solvent, a ketone solvent, a chlorinated solvent, water, and miscible mixtures thereof.

The pharmaceutically acceptable excipient used in the generation of premixes of acid salts of Nilotinib and polymorphic forms thereof may be selected from the group comprising of polysaccharides, polyvinylpyrrolidone, polyvinyl acetate, polyvinyl alcohol, polymers of acrylic acid and salts thereof, polyacrylamide, polymethacrylates, vinylpyrrolidone-vinyl acetate copolymers, C1-C6 polyalkylene glycols, and mixtures thereof.

The premixes containing acid salts and polymorphic forms thereof may be used in the formulation of oral pharmaceutical dosage forms. These oral pharmaceutical dosage forms may include additional pharmaceutically acceptable excipients and additional active pharmaceutical ingredients.

The following examples, which include preferred aspects, will serve to illustrate the practice of this invention, it being understood that the particulars shown are by way of example and for purpose of illustrative discussion of preferred aspects of the invention.

### EXAMPLES

### Example 1: Process of Nilotinib D-Malate Form C1

Nilotinib base (3 g) was dissolved in mixture of (20 V) ethanol and (40 V) MDC at 40-45° C. To the above clear solution was charged 1.14 gm (1.5 eq) of D-malic acid and dissolved at 45-50° C. The solution was concentrated under vacuum (400-200 mbar) at 50-55° C. To the residue charged (15 v) of ethanol and stirred for 16-18 hrs at Room temperature. The solids were isolated by filtration and dried in a vacuum tray drier (VTD) at 40-45° C. for 3-4 hrs to obtain 2.8 gm of the title compound.

The crystalline Nilotinib D-malate Form C1 was characterized by XRD, DSC and TGA. (FIGS. 1-3)

### Example 2: Process of Nilotinib D-Tartrate Form C1

Nilotinib base (2 g) and D-tartaric acid (0.580 gm) (1.0 eq) were suspended in a mixture of (10 v) ethanol and (10 v) MDC at Room temperature. The reaction mass was stirred for 1 hr at RT and then heated up to 50-55° C. and stirred further for 1 hr. The reaction mass was cooled to Room temperature and stirred for 2 hrs. The solids were isolated by filtration and dried in an air tray dryer (ATD) at 45-50° C. for 3 hrs to obtain 2.2 gm of the title compound.

The crystalline Nilotinib D-tartrate Form C1 was characterized by XRD, DSC and TGA. (FIGS. 4, 6 and 7)

### Example 3: Process of Nilotinib D-Tartrate Form C1

Nilotinib base (20 g) and D-tartaric acid (5.7 gm) (1.0 eq) were suspended in a mixture of (10 v) ethanol and (10 v) MDC at Room temperature. The reaction mass was stirred for 1 hr at RT and then heated up to 50-55° C. and stirred further for 15 min. The reaction mass was cooled to Room temperature and stirred for 2 hrs 30 min. The solids were isolated by filtration and dried in VTD at 45-50° C. for 4 hrs to obtain 23.8 gm of the title compound

FIG. 5 shows XRD of Nilotinib D-tartrate Form C1 crystals obtained by the process described in this example.

### Example 4: Process of Nilotinib D-Malate Form C1

Nilotinib base (20 g) was dissolved in mixture of (20 V) ethanol and (40 V) MDC at 40-45° C. To the above clear solution was charged 8.15 gm (1.60 eq) of D-malic acid and dissolved at 45-50° C. The solution was concentrated under vacuum (400-200 mbar) at 50-55° C. To the residue charged (10 v) of ethanol and stirred for 3-4 hrs at Room temperature. The solids were isolated by filtration and dried in VTD at 40-45° C. for 3-4 hrs to obtain 23.1 gm of the title compound.

The crystalline Nilotinib D-malate Form C1 was characterized by XRD, DSC and TGA. (FIGS. 1-3)

### Example 5: Process of Nilotinib D-Tartrate

D-(-) Tartaric acid (8.6 gm) (1.53 eq) was dissolved in a mixture of (26.6 v) methanol and (1.0 v) water at Room temperature. Nilotinib base (20.0 g) (1.0 eq) suspended in Tartaric acid solution and stirred for 1 hour. The reaction mass was heated to 55-60° C. and stirred for 1 hr 55-60° C. The reaction mass was cooled to Room temperature and stirred for 1 hr. The solids were isolated by filtration and dried at 39-45° C. for 12 hrs to obtain 21.6 gm of the title compound.

FIG. 5 shows XRD of Nilotinib D-tartrate crystals obtained by the process described in this example.

### Example 6: Process of Nilotinib D-Tartrate

Nilotinib base (10 g) was dissolved in a mixture of (25.0 v) MDC and (9.0 v) Methanol at 35-40° C. and cooled to Room temperature. Dissolved D-(-) Tartaric acid (4.53gm) (1.6 eq) in (4.0 v) Water and (2.0 v) Methanol. Added the D-Tartaric acid solution to Nilotinib base solution and reaction mass stirred for 4 hours at Room temperature. The solids were isolated by filtration and dried in VTD at 37-43° C. for 12 hrs to obtain 11.0 gm of the title compound.

FIG. 5 shows XRD of Nilotinib D-tartrate crystals obtained by the process described in this example.

### Example 7: Chemical Stability of Nilotinib D-Tartrate

Chemical stability of Nilotinib D-tartrate as shown in Table 1 below, indicates that Nilotinib D-tartrate was chemically more stable and exhibited better long term storage stability with significantly less degradation (% total deg products). Conditions evaluated include temperature and relative humidity (RH).

TABLE 1

| | | | 5- methyl | |
|---|---|---|---|---|
| | Time | | isomer | Total |
| Storage | points | | impurity | impurities |
| condition | Months | XRPD | (NMT 3 ppm) | (NMT 0.4%) |
| 25 ± 2° C./60 ± | 0 | Form C1 | 0.6 | 0.01 |
| 5% RH | 1 | Stable | 0.5 | NA |
| | 2 | Stable | 0.7 | NA |
| | 3 | Stable | 1.1 | 0.04 |
| 2 to 8° C. | 0 | Form C1 | 0.6 | 0.01 |
| | 1 | Stable | 0.5 | NA |
| | 2 | Stable | 0.7 | NA |
| | 3 | Stable | 1.1 | 0.05 |

Note:
NMT: Not More Than,
NA: Not Applicable

Table 1 shows no significant degradation or change in the XRPD pattern when stored at 2 to 8° C. or 25±2° C./60±5% RH, indicates that Nilotinib D-tartrate has improved thermal stability.

1-31. (canceled)

32. Nilotinib D-tartrate.

33. The nilotinib D-tartrate of claim 32, wherein the mole ratio of nilotinib to D-tartaric acid is 1:1.

34. The nilotinib D-tartrate of claim 32, in a substantially crystalline form

35. The nilotinib D-tartrate of claim 34, having an XRD pattern comprising peaks at about 6.88, 10.47, 11.24, 13.50, 18.28, and 24.17±0.2 °2θ.

36. The nilotinib D-tartrate of claim 35, wherein the XRD pattern further comprises peaks at 15.57, 19.48, 20.77, and 21.92±0.2 °2θ.

37. The nilotinib D-tartrate of claim 32, wherein the nilotinib D-tartrate is a solvate or hydrate.

38. The nilotinib D-tartrate of claim 32, having a purity of greater than 99%.

39. The nilotinib D-tartrate of claim 32, having a purity of greater than 99.5%.

40. The nilotinib D-tartrate of claim 32, comprising 0.10% or less of a 5-methyl isomer impurity, said 5-methyl isomer impurity having the structure:

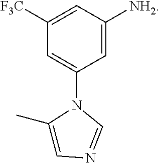 [SMILES: Cc1cncn1-c1cc(N)cc(C(F)(F)F)c1]

41. A pharmaceutical composition comprising the nilotinib D-tartrate of claim 32, and at least one pharmaceutically acceptable excipient.

42. The pharmaceutical composition of claim 41, wherein the nilotinib D-tartrate is present in an amount of 150 mg or 200 mg.

43. A method for treating chronic myelogenous leukemia in a patient in need thereof, comprising administering to the patient the nilotinib D-tartrate of claim 32.

44. The method of claim 43, wherein the chronic myelogenous leukemia is drug resistant chronic myelogenous leukemia.

45. A process for preparing nilotinib D-tartrate, comprising reacting nilotinib free base with D-tartaric acid in a solvent.

46. The process of claim 45, wherein the mole ratio of nilotinib free base to D-tartaric acid is 1:1.

47. The process of claim 45, wherein the solvent comprises an alcohol, an ether, ketone, an ester, a chlorinated solvent, a hydrocarbon, a nitrile, or a mixture thereof.

48. The process of claim 45, wherein the solvent comprises a polar, aprotic solvent selected from the group consisting of dimethyl formamide, dimethyl acetamide, dimethyl sulfoxide, N-methyl-2-pyridone, N-methyl-2-pyrrolidone, 1-methyl-2-pyrolidinone, and mixtures thereof.

49. The process of claim 45, comprising dissolving nilotinib free base in isolated form in the solvent, and then adding D-tartaric acid.
